(19) United States
(12) Patent Application Publication
(10) Pub. No.: US 2003/0143109 A1
(43) Pub. Date: Jul. 31, 2003

(54) Methods for treating surfaces

(76) Inventors: Darren McKnight, Centreville, VA (US); William Felder, Lawrenceville, NJ (US); Charles Berman, East Windsor, NJ (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/059,258

(22) Filed: Jan. 31, 2002

(51) Int. Cl. A61L 2/00

(52) U.S. Cl. 422/29; 422/28; 422/37; 422/23

(57) ABSTRACT

Disclosed are methods for sterilizing or disinfecting a surface, which comprise applying an electrostatically-charged aerosol of a disinfecting solution onto said surface and allowing said aerosol to remain in contact with said surface for a time sufficient to achieve the desired degree of sterilization or disinfection.

### CROSS-REFERENCES TO RELATED APPLICATIONS

Not applicable.

### STATEMENT REGARDING FEDERALLY SPONSORED RESEARCH

Not applicable.

### BACKGROUND OF THE INVENTION

1. Field of the Invention

The present invention relates to methods for disinfecting or sterilizing surfaces via electrostatically charged disinfecting solutions.

2. Description of the Related Art U.S. Pat. No. 4,704,942 is directed to a method of defending against a warfare cloud of toxic aerosol, which utilizes a charged defensive aerosol which may contain a disinfectant. The defensive aerosol is said to have a charge of at least 10,000 volts, and preferably at least 100,000 volts.

There remains a need for methods for treating a wide variety of surfaces to reduce the concentration of viable microorganisms which may be present thereon.

### BRIEF SUMMARY OF THE INVENTION

In one aspect, the present invention relates to a method for sterilizing or disinfecting a surface, which comprises:

a) forming an electrostatically-charged aerosol of a disinfecting solution;

b) applying said aerosol onto said surface; and

c) allowing said aerosol to remain in contact with said surface for a time sufficient to achieve the desired degree of sterilization or disinfection.

### BRIEF SUMMARY OF THE DRAWINGS

FIG. 1 is a depiction of an aspect of the operation of the present invention.

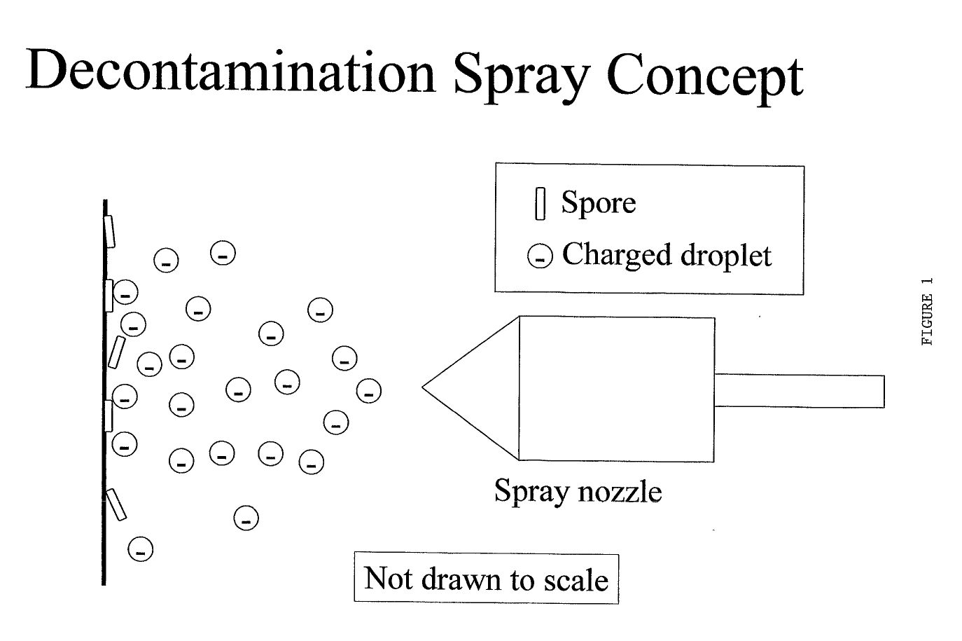

### DETAILED DESCRIPTION OF THE INVENTION

The present methods are useful to disinfect or sterilize various surfaces. The term “sterilization” as used herein generally denotes the process of eliminating all viable microorganisms from a surface, including the spores of the microorganism. The term “disinfection” as used herein generally refers to the process of destroying, or sometimes merely reducing, the potential infectivity of the material and does not necessarily imply the removal of all viable microorganisms and their spores.

The present invention is effective against a wide range of microorganisms and their spores. In a preferred embodiment, the present invention may be used to treat surfaces containing, or suspected to contain, spores of Bacillus anthracis.

A wide range of suitable disinfecting solutions may be used in connection with the present invention. Basically, any solution suitable to disinfect a surface can be used. Preferred are biological oxidants. Particularly preferred are halogen containing compounds, such as, for example, chlorine dioxide, bromine oxide, bromine chloride, monochloroamine, bromic acid, hypochlorous acid, chlorates, chlorites, hypochlorites, iodine monochloride, iodine trichloride iodine monobromide, etc. Combinations of two or more suitable compounds are within the scope of the present invention. Especially preferred are chlorine dioxide and hypochlorous acid, either individually or in combination.

The present invention entails the formation of an electrostatically-charged aerosol of the disinfecting solution. How to do so would be readily apparent to one of ordinary skill. Preferably, such can be accomplished by the use of an electrostatic sprayer, many of which are commercially available. One preferred model is sold by Electrostatic Spraying Systems, Inc. and includes a MaxCharge™ induction electrostatic nozzle.

Preferably, the aerosol contains small charged droplets, having diameters ranging from about 10 microns to about 80 microns, more preferably from about 30 microns to about 40 microns. A suitable degree of charge is from about one to about ten millicoulombs per milliliter of solution, preferably about five millicoulombs per milliliter of solution, which generally corresponds to an applied voltage of about 1000 volts or lower.

The aerosol may be applied to the surface to be sterilized or disinfected at any suitable rate. A preferred rate of application is from about 0.1 to about 5 ft² per second, with a rate of from about 0.5 to about 2 ft² per second being particularly preferred. Preferably the application rate and solution concentration are adjusted such that the wetting of the surface to be treated is minimized. Indeed, the surface may not even feel wet to the touch. As a result, a wide variety of surfaces may be treated, including walls, floors, paper, computers, electrical components, etc.

The aerosol should be allowed to remain on the surface to be treated for a time sufficient to achieve the desired sterilization or disinfection, which will depend on the particular solution used. In general, dwell times of about 2 minutes and above should be sufficient.

Surfaces that have been contaminated from the introduction of a large sample of biological pathogen will have some random distribution of spores adhering to them. It is believed that the spray will be used to first attach these spores to the surface by the first fine misting of the spray to reduce further cross-contamination. Additional applications of the spray will provide more solution on the site to produce a microenvironment of the spore with high relative humidity and also a high concentration of oxidative solution that will kill the spores. This treatment provides the logistically easiest means to provide a high relative humidity and high concentration of oxidative material with minimal material degradation and high effectiveness.

1. A method for sterilizing or disinfecting a surface, which comprises:
a) forming an electrostatically-charged aerosol of a disinfecting solution;
b) applying said aerosol onto said surface; and
c) allowing said aerosol to remain in contact with said surface for a time sufficient to achieve the desired degree of sterilization or disinfection.

2. The method of claim 1, wherein the aerosol comprises droplets having diameters in the range of from about 10 microns to about 80 microns.

3. The method of claim 2, wherein the droplets have a charge of from about one to about ten millicoulombs per milliliter of solution.

4. The method of claim 1, wherein the disinfecting solution comprises an aqueous solution of a biological oxidant.

5. The method of claim 4, wherein the biological oxidant comprises a halogen containing compound.

6. The method of claim 5, wherein the halogen containing compound is selected from the group consisting of chlorine dioxide, bromine oxide, bromine chloride, monochloroamine, bromic acid, hypochlorous acid, chlorates, chlorites, hypochlorites, iodine monochloride, iodine trichloride, iodine monobromide, and combinations thereof.

7. The method of claim 6, wherein the halogen containing compound is hypochlorous acid.

8. The method of claim 1, wherein said aerosol is applied to said surface at the rate of from about 0.1 to about 5 ft² per second.

9. The method of claim 8, wherein said aerosol is applied to said surface at the rate of from about 0.5 to about 2 ft² per second.

10. The method of claim 1, wherein said treatment is effective to at least reduce the potential infectivity of any viable microorganisms, including spores thereof, residing on said surface.

11. The method of claim 10, wherein said treatment is effective to at least reduce the potential infectivity of any spores of Bacillus anthracis.

12. The method of claim 1, wherein the aerosol remains in contact with said surface for at least about 2 minutes.
